(19) United States
(12) Patent Application Publication
(10) Pub. No.: US 2022/0096509 A1
(43) Pub. Date: Mar. 31, 2022

(54) Reformulating Hospital Gentamicin to reduce the risk of hearing loss while maintaining antimicrobial activity

(71) Applicant: The Board of Trustees of the Leland Stanford Junior University, Sanford, CA (US)

(72) Inventors: Mary E. O'Sullivan, San Francisco, CA (US); Anthony J. Ricci, Palo Alto, CA (US); Alan G. Cheng, Palo Alto, CA (US); Robert J. Greenhouse, Newark, CA (US); Markus E. Huth, Murzelen (CH); Randy Lin, Santa Clara, CA (US)

(21) Appl. No.: 17/425,591

(22) PCT Filed: Feb. 7, 2020
(86) PCT No.: PCT/US2020/017179

§ 371 Date: Jul. 23, 2021

Related U.S. Application Data
(60) Provisional application No. 62/803,228, filed on Feb. 8, 2019.
(60) Provisional application No. 62/893,342, filed on Aug. 29, 2019.

(51) Int. Cl. A61K 31/7036; A61P 31/04

(52) U.S. Cl. CPC A61K 31/7036; A61P 31/04

(57) ABSTRACT

Reformulations of gentamicin for treatment of infections are provided with the goal to reduce current side effects such as ototoxicity, while maintaining antimicrobial activity. The reformulation is a mixture of three of the C-components of gentamicin: C1a, C2a and C2b, while excluding as much as possible, C1 and C2. Specifically, to the objective of this invention a mixture is formulated having gentamicin C1a ranging from 10-30%, gentamicin C2a ranging from 0-30%, and gentamicin C2b ranging from 40-90%, where the mixture totals a 100% of the gentamicin C-components, defined as C1, C1a, C2, C2a and C2b only. The formulations of this invention increase the probability of a more favorable outcome for the patients exposed to gentamicin, i.e., reduced risk of the side-effect of hearing loss.

### FIELD OF THE INVENTION

This invention relates to formulations of hospital gentamicin to reduce side effects.

### BACKGROUND OF THE INVENTION

Aminoglycosides are a class of antibiotics classified as critical by the World Health Organization. In the UK and the USA, the aminoglycoside gentamicin is commonly given to children in neonatal intensive care units where there is a high incidence of hearing loss. This incidence of hearing loss in neonatal intensive care units is ten times higher than in the well-baby nursery population. Most ear-related safety studies of gentamicin toxicity to the ear (ototoxicity) regard it as a single entity. However, gentamicin is a mixture of over 20 chemical components.

Five C-components make up 90% or greater of the gentamicin mixture and the remaining 10% or less are impurities. The United States Pharmacopeia (USP) requires specific ranges for each C-component (Table 1) and the maximum limit for each impurity is 3%. Three C-components is the minimum number of components required for hospital gentamicin.

This gentamicin mixture is a product of a fermentation process by the Micromonospora bacterial species. It is unclear why a gentamicin mixture versus an individual compound is in clinical use and on what basis the current USP guidelines are based. The mixture may be in use because it has been in use since the 1960s (grandfathered in through the FDA in the USA). The USP ranges for the C-components may also account for the variation that occurs between different batches of Micromonospora fermentations (Table 1).

The ototoxicity of some individual C-components of hospital gentamicin was previously reported. The ototoxicity of the gentamicin C2b component has not been previously reported. Other, individual C-components were studied in different animal models including zebrafish, mice, guinea pigs, rats and humans. These reports include in vitro and in vivo models, and different methods to assess ototoxicity. However, there is discordance between the results reported, and in many studies chemical validation (NMR and HPIC-MS composition and purity data analysis) of the individual components studied has not always been published. Knowledge of these details is important because subtypes have been mis-assigned in the past and specialized conditions are required for detection and chemical separation of C2, C2a and C2b which have the same molecular weight.

The objective of this invention was to define new formulations for gentamicin components to reduce side effects such as the risk of hearing loss, while maintaining antimicrobial activity.

TABLE 1
Current calculations of the composition of gentamicin
according to USA Pharmacopoeia (USP) and Europe Pharmacopoeia
(EP) standards. The calculation of the composition for
EP and USP slightly differ. In the EP, the sum of C2,
C2a and C2b is used, in the USP the sum of C2 +
C2a and the sum of C2b + C1 are used.

| | Current Limits | Current Limits |
|---|---|---|
| Gentamicin | USP | EP |
| C1a | 10-35% | 10-30% |
| C2 | 25-55% | 35-55% |
| C2a | | |
| C2b | 25-50% | |
| C1 | | 25-45% |

### SUMMARY OF THE INVENTION

The present invention provides reformulations of gentamicin for treatment of infections with the goal to reduce current side effects while maintaining antimicrobial activity. The reformulation is a mixture three C-components of gentamicin: C1a, C2a and C2b. Specifically to the objective of this invention a mixture is formulated to have gentamicin C1a ranging from 10-30%, gentamicin C2a ranging from 0-30%, and gentamicin C2b ranging from 40-90%, where the mixture totals a 100% of the gentamicin C-components, defined as C1, C1a, C2, C2a and C2b only. In one variation, this formulation is defined such that the combination of the C2a and the C2b is larger than 55%. This reformulation embodiment could be varied as another embodiment by adding to the mixture gentamicin C1 and/or C2 both ranging from 0-10%. In a further variation, this formulation is defined as another embodiment such that the combination of the C2b, C2a and C2 is larger than 55%. In still a further variation, this formulation is defined as another embodiment such that the combination of the C2a and C2 is less than 25%. In still a further variation, this formulation is defined as another embodiment such that the combination of the C2b, C2a and C2 is less than 35%. In a further variation of the invention, is to define the formulation as a mixture consisting essentially of C1a, C2a and C2b with the specific ranges outlined herein.

In yet another variation, the formulation of gentamicin for treatment of infections is defined as gentamicin C2b ranging from 90-100% of a total percentage of gentamicin C-components, defined as C1, C1 a, C2, C2a and C2b, and impurities ranging from 0-10% in total having the mixture at 100%.

The formulations of this invention provide that greater quality control over hospital gentamicin will enable standardization of treatment for patients, thus increasing the probability of a more favorable outcome for the patient. For example, gentamicin C2 is allowed to be in hospital bottles up to 55% at present, however data presented herein indicates this drug component is the most ototoxic component and a bottle with high C2 content clearly presents a much higher risk to patients than our current formulation (i.e., based on data by the inventors increased C2 content increases the risk of hearing loss to patients). In other words, a reformulated hospital bottle with C1a, C2a, and C2b can replace the current treatment regimens with fewer side effects and superior outcomes in patients.

A less ototoxic gentamicin reformulation could also be beneficial to a subset of patients reported to experience hearing loss following a single dose of this antibiotic, i.e. patients with the m.1555A>G or m. 1494C>T mitochondrial DNA mutations. Likewise, a less ototoxic reformulation could be beneficial to studies and clinics using aminoglycosides as a therapy for premature stop codon diseases.

### BRIEF DESCRIPTION OF THE DRAWINGS

FIG. 1A-E show according to exemplary embodiments of the invention how we leveraged the variation in gentamicin biosynthesis to isolate and purify individual gentamicin C-components according to an exemplary embodiment of the invention. (1A) The biosynthesis pathway of the gentamicin C-components in Micromonospora species. The gentamicin components making up >90% of the hospital bottle are shown in grey boxes. For the gentamicin C-components, the following color scheme is used throughout C1a=blue, C2b=purple, C2a=green, C2=cyan, C1=orange. (1B) Gentamicin is a three-ringed aminoglycoside molecule. It contains a central 2-deoxystreptamine with an ammonium group on either side of the deoxy carbon and added at position 4 to ring I, and at position 6 to ring III. (1C) Variation between fermentation reactions results in different quantities of the C-components. HPIC traces from 5 vendors are shown. Vendor D and Vendor D were used for the purification of the C-components of gentamicin. This screen allowed us to identify different lots of commercial gentamicin to find the optimal mixture to pull out each component rather than using a single lot. (1D) HPIC traces of gentamicin C1a, C2, C2b, C2a and C1a demonstrating composition of matter (comparative diagrams of FIG. 1D and FIG. 1E are adjacent to each other). Gentamicin components were additionally validating using ¹³C and ¹H NMR spectroscopy. (1E) The C-components of gentamicin differ at the 5′ and 6′ position on ring I. The differences between the C-components are shown.

FIG. 2A-D show that the C1, C1a, C2, C2a, and C2b components of gentamicin have similar antimicrobial breadths and potencies to a hospital bottle, and that the impurities have variable breadths and potencies according to an exemplary embodiment of the invention. (2A) The antimicrobial breadths of the C-components relative to a hospital bottle, where breadth is defined as having a MIC value ≤4μg/ml. The colors represent different species, the numbers in the columns refer to the number of individual strains inhibited, where black=S. aureus, grey=E. coli, blue=K. pneumoniae, white=P. aeruginosa. The hospital bottle inhibits 35/40 strains tested, with the C-components inhibiting 31-35 strains. (2B) The MIC values for each strain (diamond symbols) split by species, where Hospital=red, C1=orange, blue=C1a, cyan=C2, green=C2a, purple=C2b (grey scale only). Filled symbols indicate strains with antimicrobial resistance. The box indicates the 25-75 percentiles, stars indicate geometric means, *=P<0.05, **=P<0.01 tested using Mann Whitney U Non-Parametric analysis. The C1 component is less potent than the hospital bottle against S. aureus strains (P<0.05, Mann Whitney U Test, n=10 strains). There are also some differences between individual components in S. aureus and P. aeruginosa. (2C) The antimicrobial breadths of the impurities relative to a hospital bottle. The hospital bottle inhibits 35/40 strains tested, with the impurities Gent B, Gent A, Gent X and G418 inhibiting 15-21 strains. One impurity, Sisomicin inhibited 35/40 strains. (2D) The MIC values for each strain split by species. The impurities Gent B, Gent A, Gent X and G418 are all significantly less potent than the hospital bottle in all species tested (P<0.05, Mann Whitney U Test, n≥8 strains). One impurity, Sisomicin was equi-potent to the hospital against E. coli and K. pneumoniae (P>0.05, Mann Whitney U Test, n≥strains), and was more potent than the hospital against S. aureus and P. aeruginosa (P<0.05, Mann Whitney U Test, n-8 strains).

FIG. 3A-E show the C1, C1a, C2, C2a, and C2b components of gentamicin, as well as the impurities, have different ototoxicities according to an exemplary embodiment of the invention. (3A) Schematic of the experimental method used to study ototoxicity in vitro using post-natal day 5 ear-explants containing sensory hair cells in the organ of corti. (3B) Representative images of undamaged and damaged organ of corti tissues. Scale bar: 25 μm. (3C) Dose responses of the individual C-components relative to a hospital bottle. Dose response curves represent hair cell survival in the mid-apical region of the cochlea. C2 is more ototoxic and C2b less ototoxic than the hospital bottle (P<0.05, F-test, n>5 per individual data point; EC50±SE, C2=402±38 μM, Hos=556±54 μM, C2b=1198±114 μM). Data are shown as Mean±SD, n>5 for each data point. Logarithmic function best fits were used for each data set. (3D) Dose responses of the impurities relative to a hospital bottle. Sisomicin is more ototoxic than the hospital, and geneticin (G418) is more ototoxic at lower doses than the hospital but requires a higher dose to kill all hair cells than the hospital (P<0.05, F-test, n>5; EC50±SE, Sisomicin=212±7 μM, Hos=556±54 μM). (3E) Logarithmic function best fits estimate the EC50s of all components except Geneticin. For geneticin a bi-phasic function was used and the EC50s of each phase are shown.

FIG. 4A-D shows it is possible to alter the ototoxicity of the mixture without compromising antimicrobial activity, according to an exemplary embodiment of the invention. (4A) A formulation of the gentamicin mixture without impurities by adding together individual gentamicin C-components (Mix 1). The percentage composition of this bottle was 53% gentamicin C1, 24% C1a, 15% C2, 6% C2a, and 2% C2b, wherein the mixture totals a 100% of the gentamicin C-components only. This was determined from preliminary analysis of a hospital bottle (data not shown). (4B) (i) C2 is more ototoxic than Mix 1 (P>0.05, F-test, n>5; EC50±SE, Mix 1=670±10 μM, C2=402±38 μM) (ii) We removed C2 from the mixture and we kept the ratio of the remaining components the same (No C2). (iii-iv) Removal of C2 from the mixture did not affect antimicrobial breadth or potency (P>0.05, Mann Whitney U Test, n=40 strains). (4C) (i) C1 has the same ototoxicity as Mix 1 (P>0.05, F-test, n>5; Mix 1=670±10 μM, C1=755±60 μM) (ii) We removed C1 from the mixture and we kept the ratio of the remaining components the same (No C1). (iii-iv) Removal of C1 from the mixture did not affect antimicrobial breadth or potency (P>0.05, Mann Whitney U Test, n=40 strains) (D) (i) We next sought to replace the C2 and C1 content with C2b (Mix 2). (ii-iii) Replacement of C2 and C 1 with C2b did not impair antimicrobial breadth or potency (P>0.05, Mann Whitney U Test, n=40 strains). (iv) Mix 2 has reduced ototoxicity (P<0.05, F-test, n>5; EC50±SE, Mix 1=670±10 μM, Mix 2=1148±52 μM).

FIG. 5 shows the secondary structures of the C1, C1a, C2, C2a, and C2b components of gentamicin.

### DETAILED DESCRIPTION

For the purposes of this invention, i.e. the reformulation of gentamicin to reduce side effects while maintaining antimicrobial activity, data was collected on the antimicrobial and ototoxic potencies of each of the individual components of hospital gentamicin. All five of the individual gentamicin C-components had not been tested previously. The following results were obtained.

Purifying Individual Gentamicin C Components from Naturally Occurring Mixtures

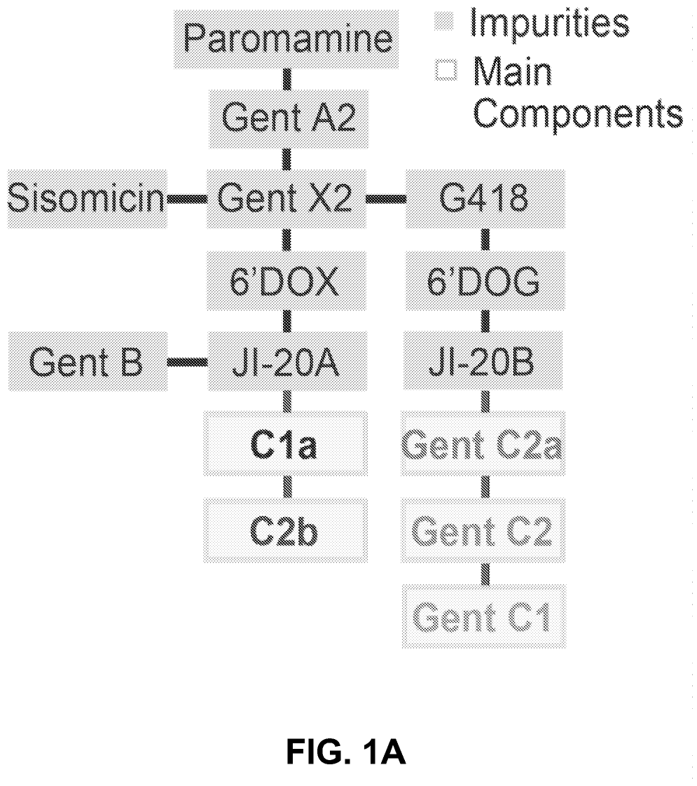

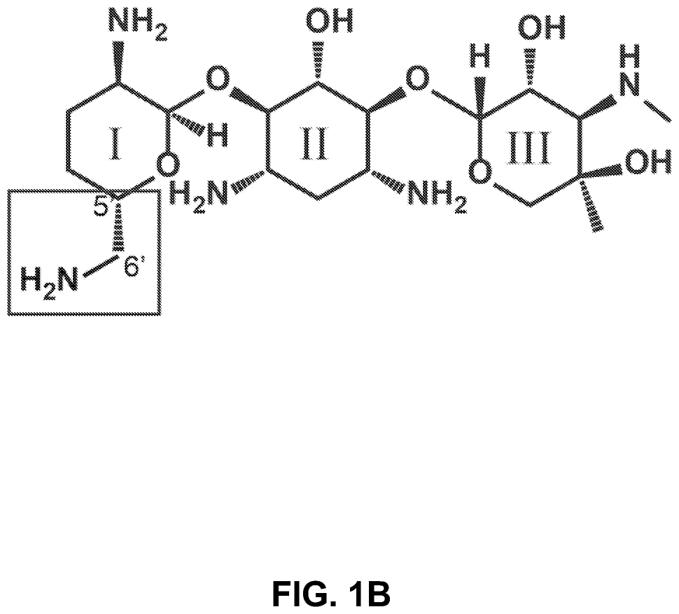

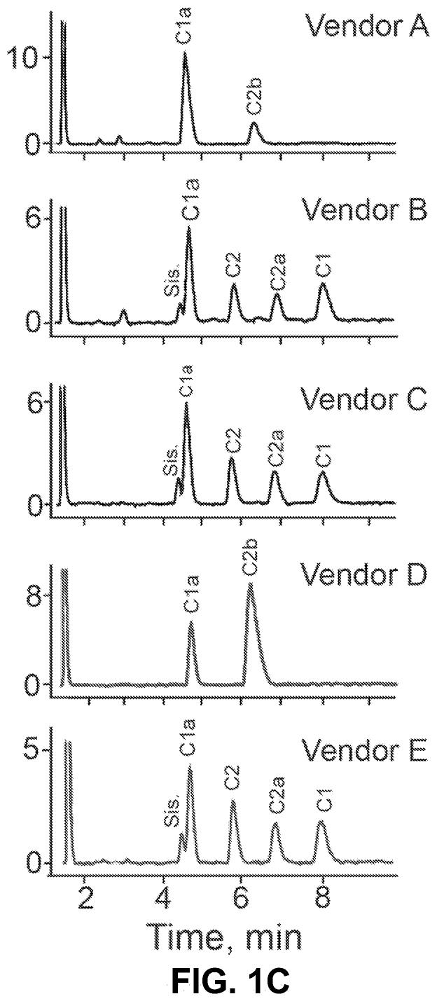

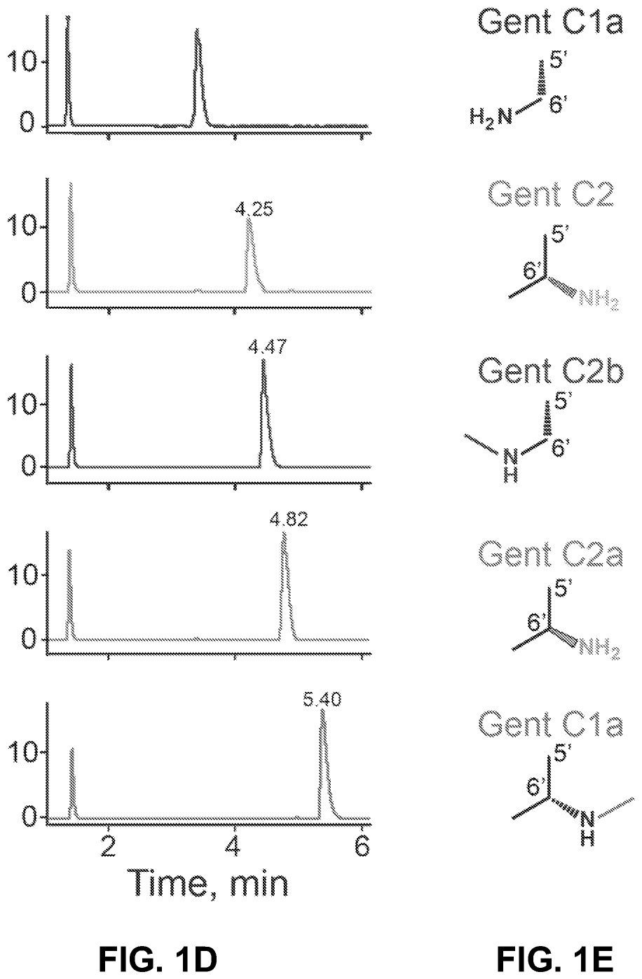

Gentamicin is a naturally occurring mixture. For clinical application, the total gentamicin mixture must be made up of >90% C-components. The biosynthesis pathway of the impurities and the gentamicin C-components has been recently delineated (FIG. 1A). Notably, there is a branchpoint in the biosynthesis pathway at gentamicin X2 that can have a major impact on the quantities of gentamicin C-components in the final product (FIG. 1A).

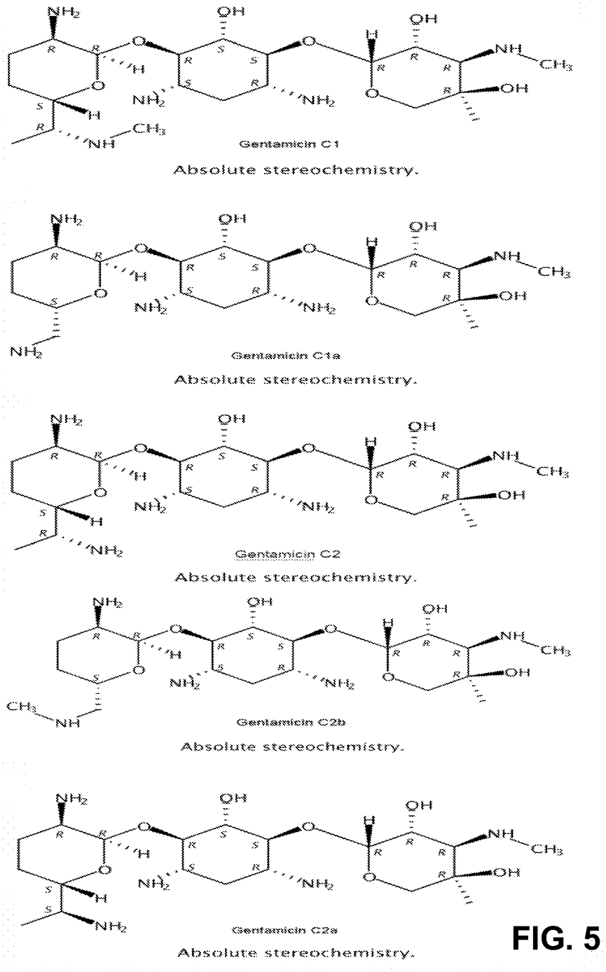

Gentamicins are three-ringed aminoglycoside molecules. The main components of gentamicin differ only at the 5′ and 6′ positions on ring I (FIG. 1B). Separation of gentamicin components is technically challenging due to the chemical similarities between C2, C2a and C2b. For the purposes of this invention, the composition of gentamicin mixtures was examined from five commercial vendors with the goal of identifying batches of gentamicin that had only C1a and C2b, and batches of gentamicin that had no C2b (FIG. 1C). Using these batches as starting materials has not been reported previously and made separation technically less challenging and amenable to gram scale preparation of individual components. The 5 main components of gentamicin were purified and validated using HPIC-MS (FIG. 1D) and NMR. The chemical structure of the gentamicin C-components is different at the 5′ and 6′ sites on Ring I (FIG. 1E and FIG. 5).

The Main Components of Gentamicin have Similar Antimicrobial Breadths and Potencies to the Gentamicin Mixture

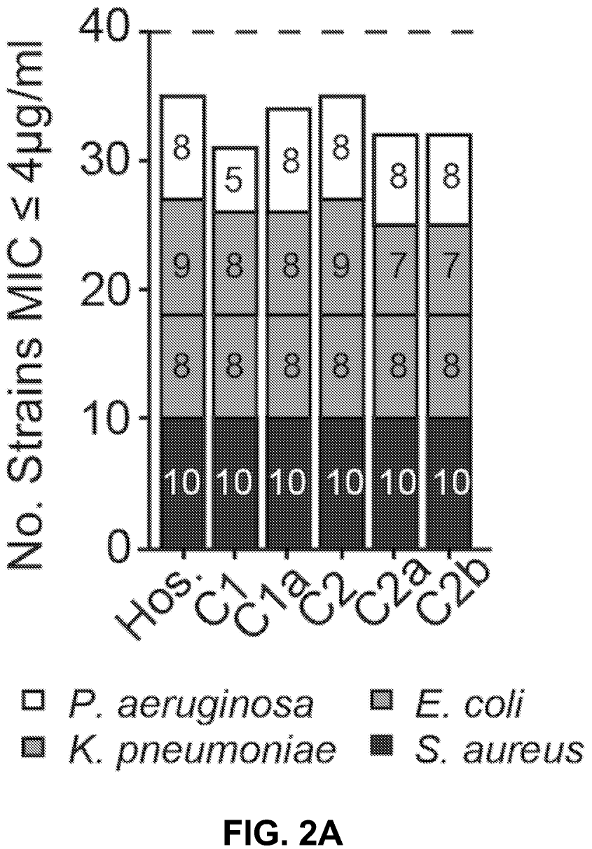

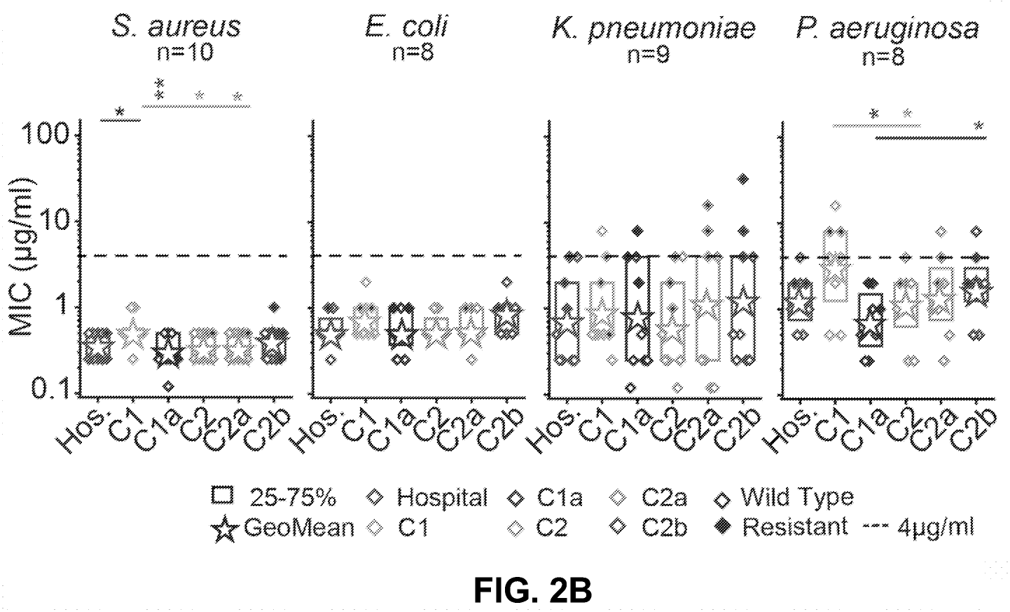

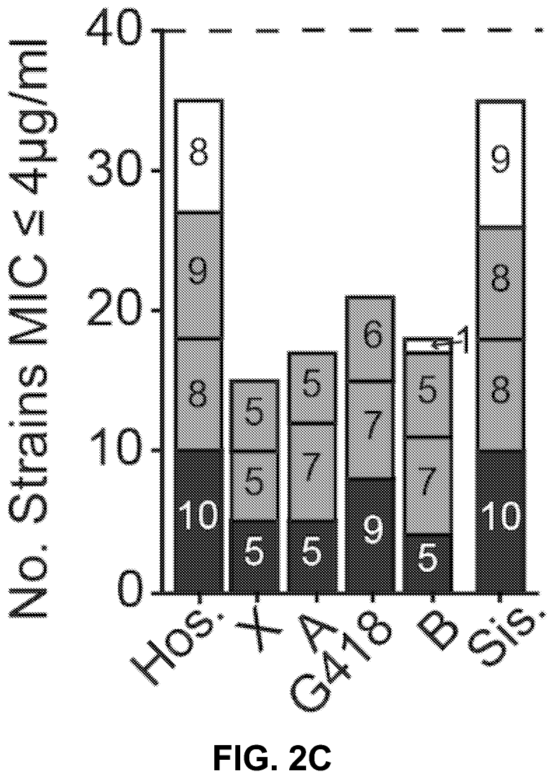

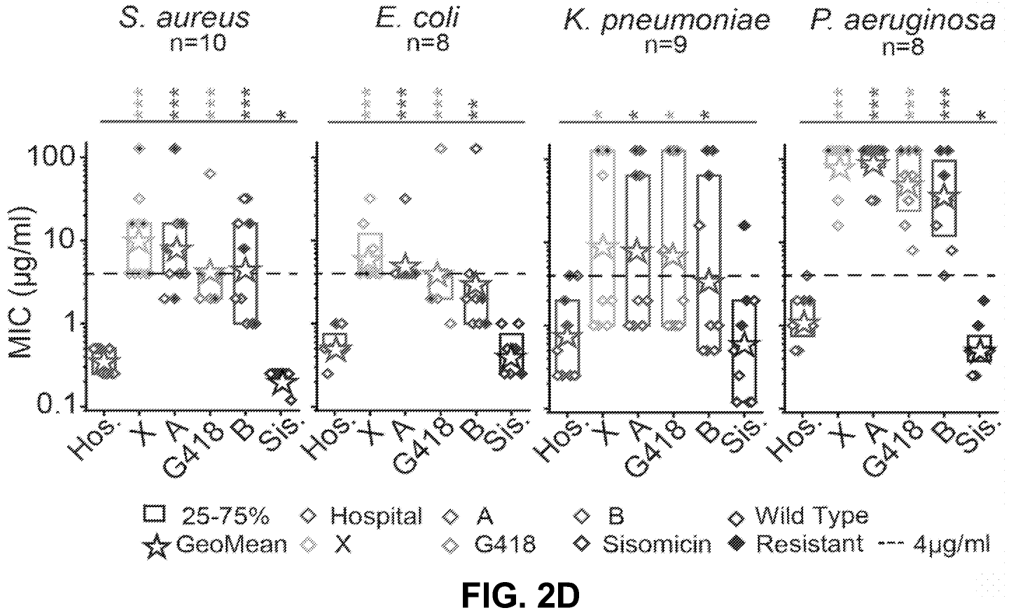

The antimicrobial activity of the main components of gentamicin were studied relative to a single hospital bottle (gentamicin mixture) from Stanford University Hospital. It was found that all five main components have similar antimicrobial breadths to the mixture (FIG. 2A). The hospital bottle inhibits 35/40 strains and each individual component inhibits from 31-35 strains (FIG. 2A). The strains inhibited are similar across gentamicin components. When we examined antimicrobial dose to inhibit (potency), we observed the C1 component to be less antimicrobial than the hospital bottle against S. aureus (P<0.05, Mann Whitney U Test, n=10 strains, Geomean Hos=0.35 μg/ml, Geomean C1=0.53 μg/m1) (FIG. 2B). Some differences between the individual components were detectable, e.g. the C1a component is more antimicrobial than the C1 component against S. aureus and P. aeruginosa strains (Mann Whitney U Test, n≥8 strains, P<0.05; S. aureus Geomean C1a=0.29 μg/ml, S. aureus Geomean C1=0.54 μg/ml, P. aeruginosa Geomean C1a=0.71 μg/ml, P. aeruginosa Geomean C1=1.52 μg/ml).

The Main Components of Gentamicin have Different Ototoxicities

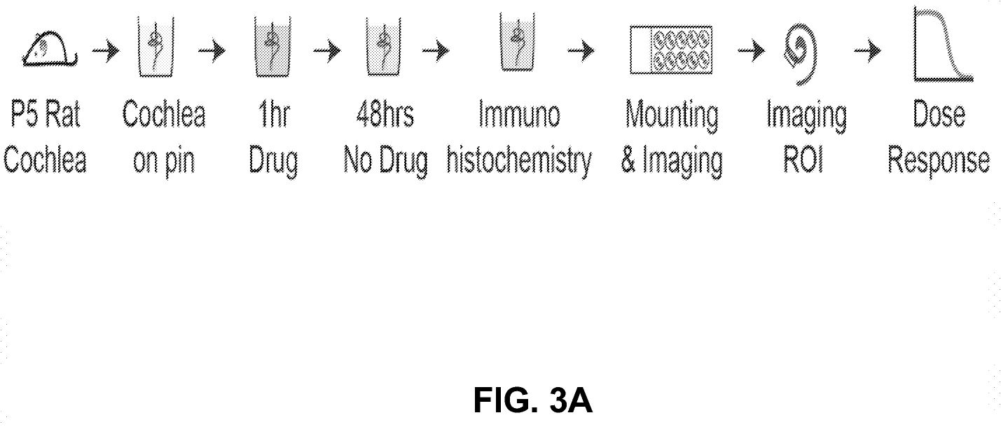

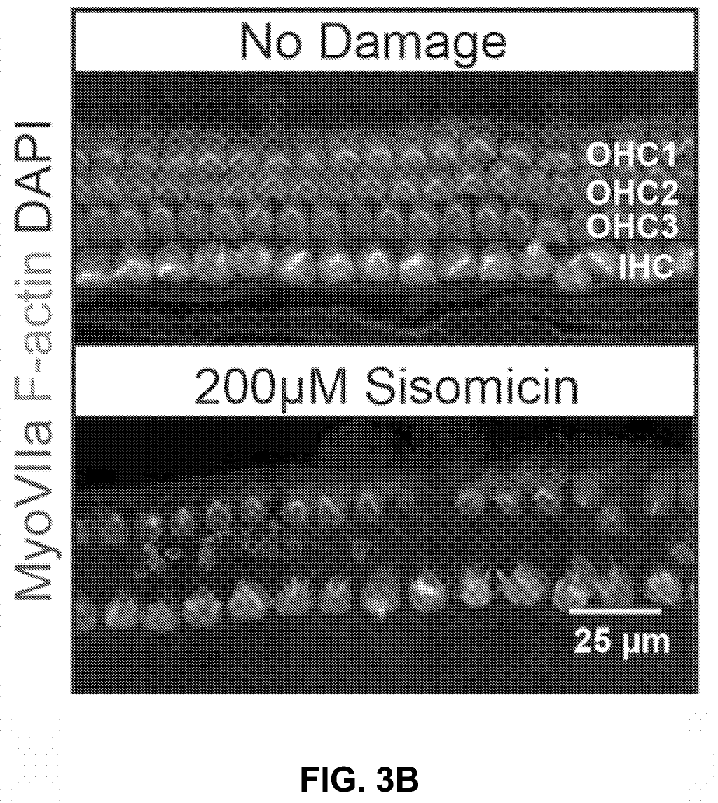

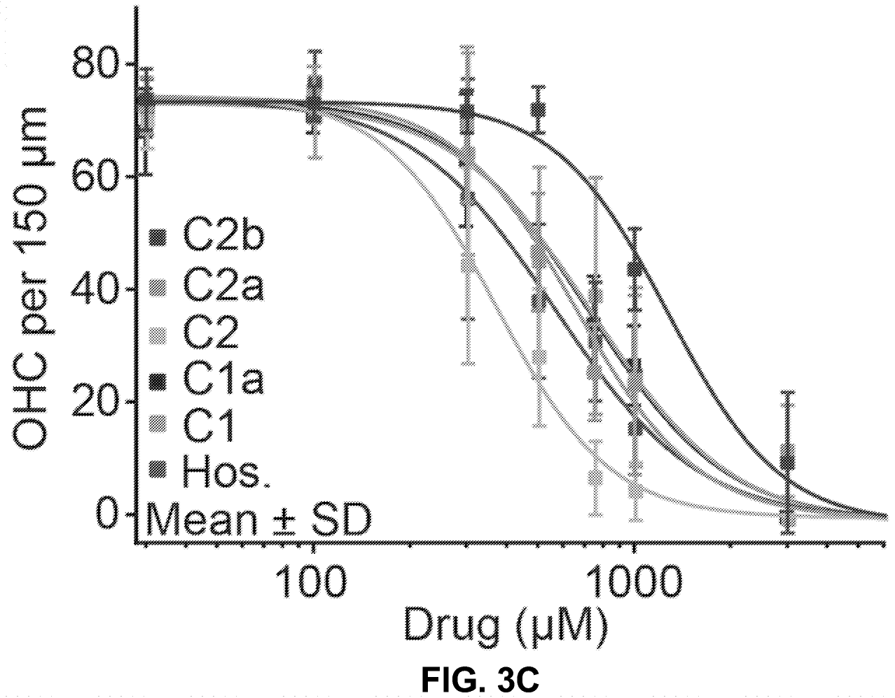

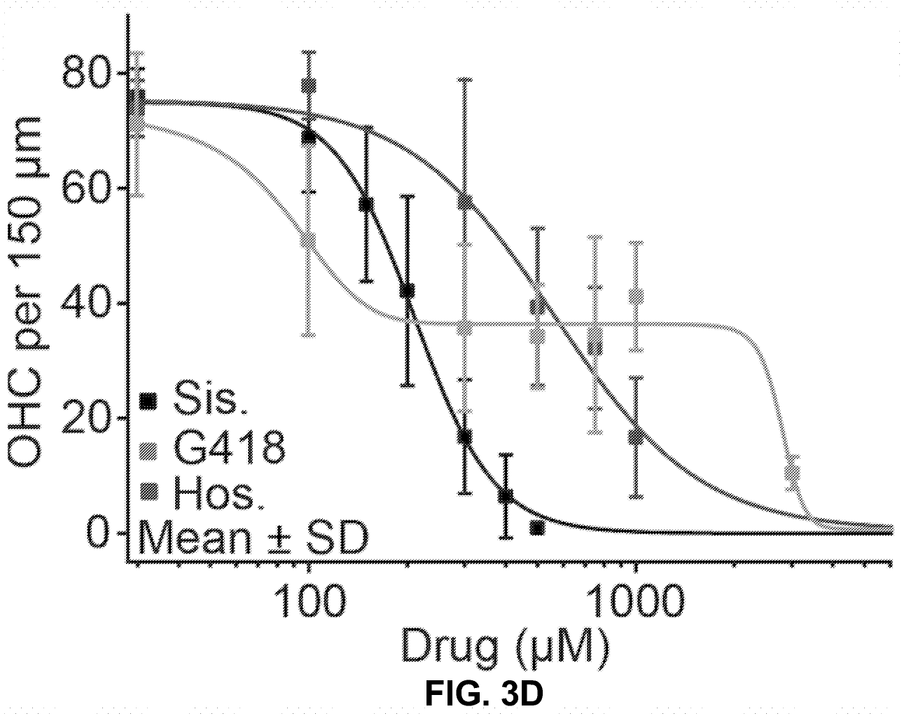

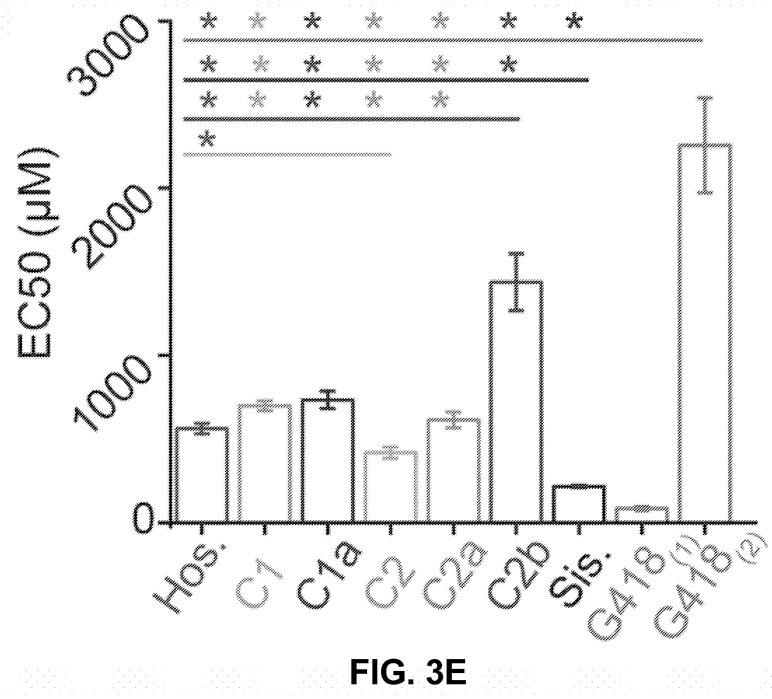

The toxicity of gentamicin components to rat cochlear hair cells (cells responsible for converting the mechanical sound stimulus into a chemical signal) was examined using a medium throughput in vitro experimental protocol FIGS. 3A & B. Each main component was compared to a hospital bottle (FIG. 3C). C2 was identified to be more ototoxic than the hospital bottle and C2b is less ototoxic than the hospital bottle (P<0.05, F-test, n>5 per individual data point; EC50±SE, C2=402±38 μM, Hos=556±54 μM, C2b=1198±114 μM) (FIG. 3B). The C1, C2a and C1a components are not significantly different from the hospital bottle (P>0.05, F-test, n>5; EC50±SE, Hos=556±54 μM, C1=755±60 μM, C1a=816±18 μM, C2a=614±69 μM) (FIG. 3B).

Drug Design—Developing a Three-Component Mixture to Reduce Ototoxicity Without Compromising Antimicrobial Activity

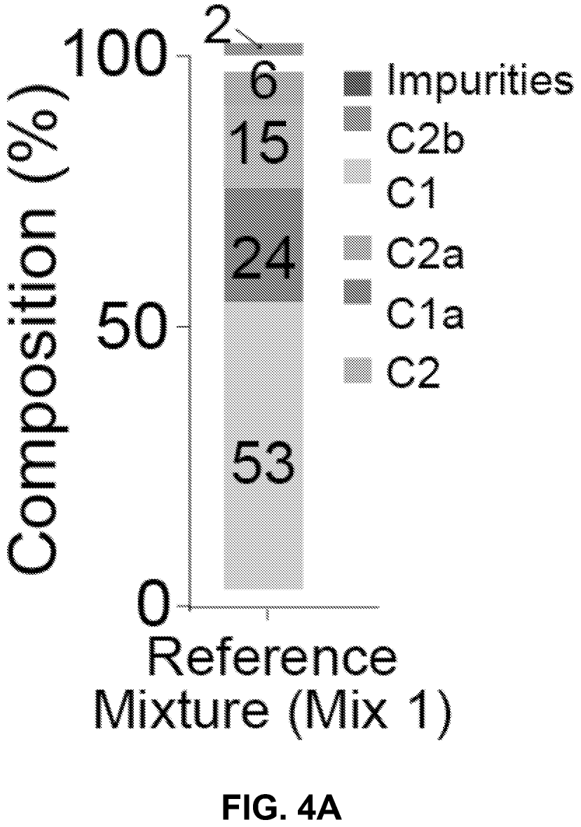

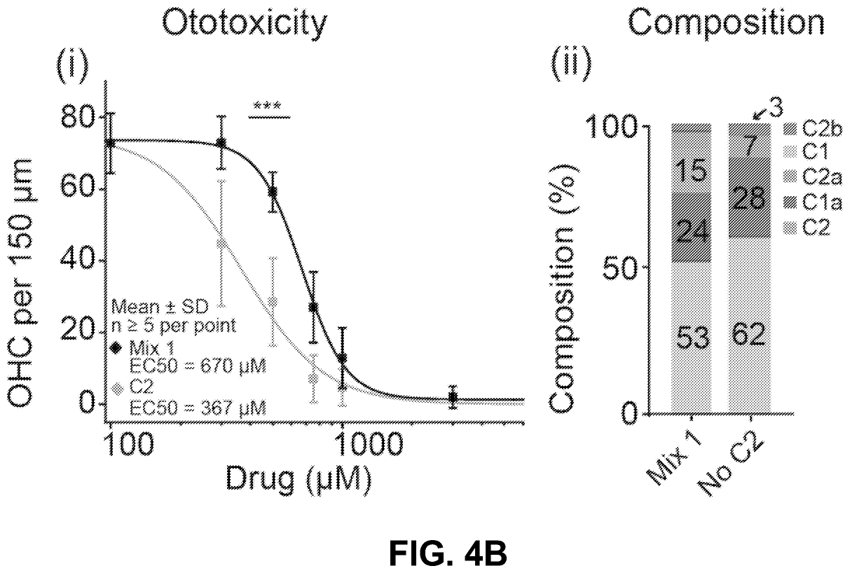

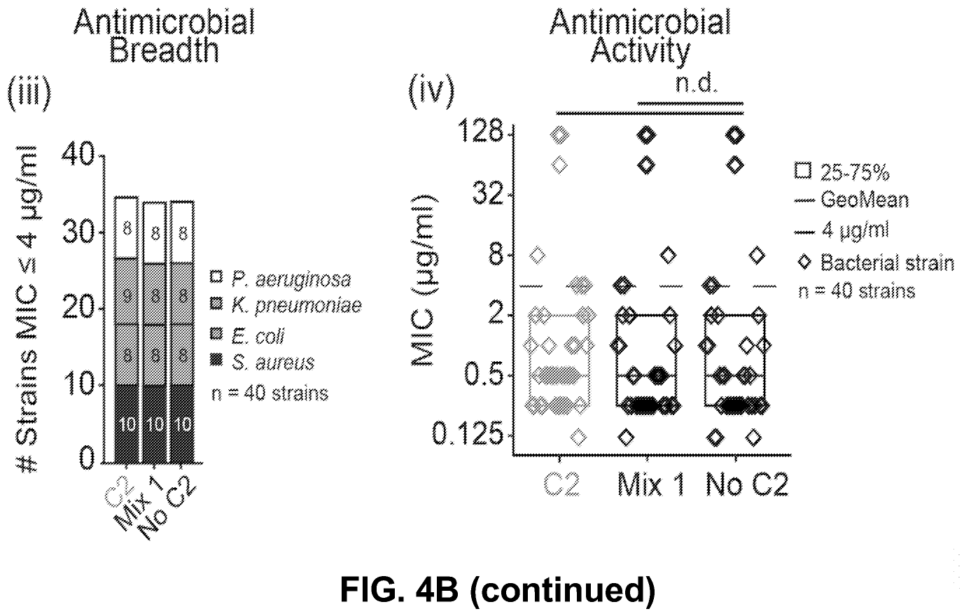

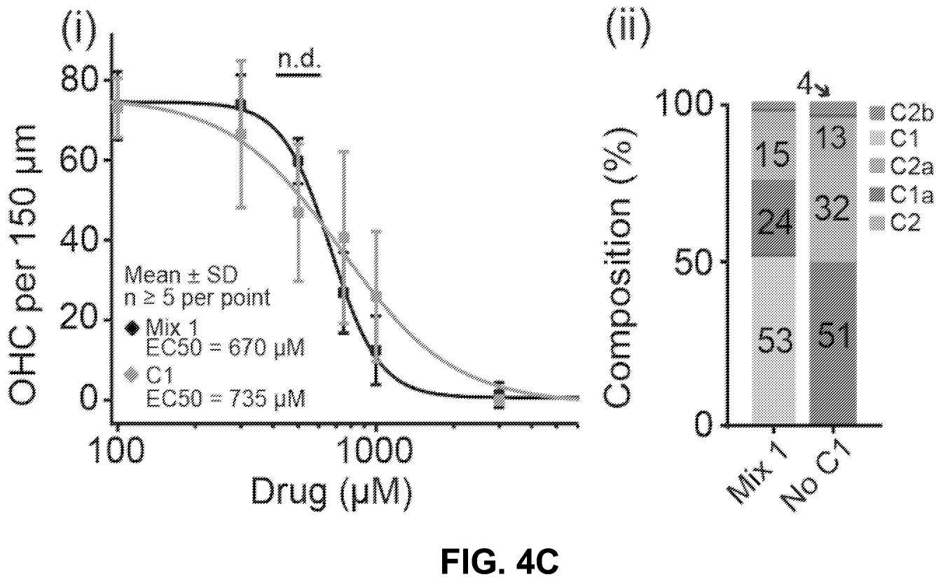

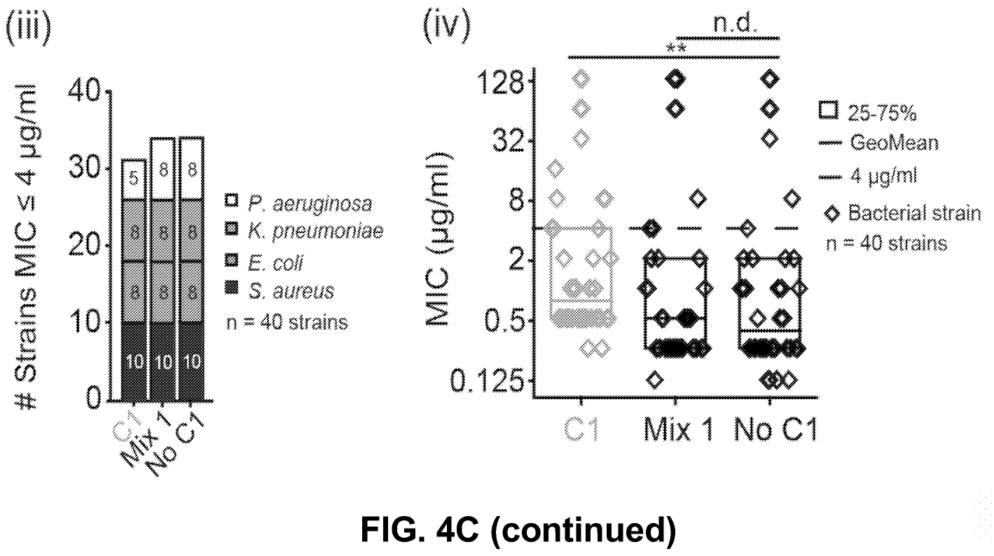

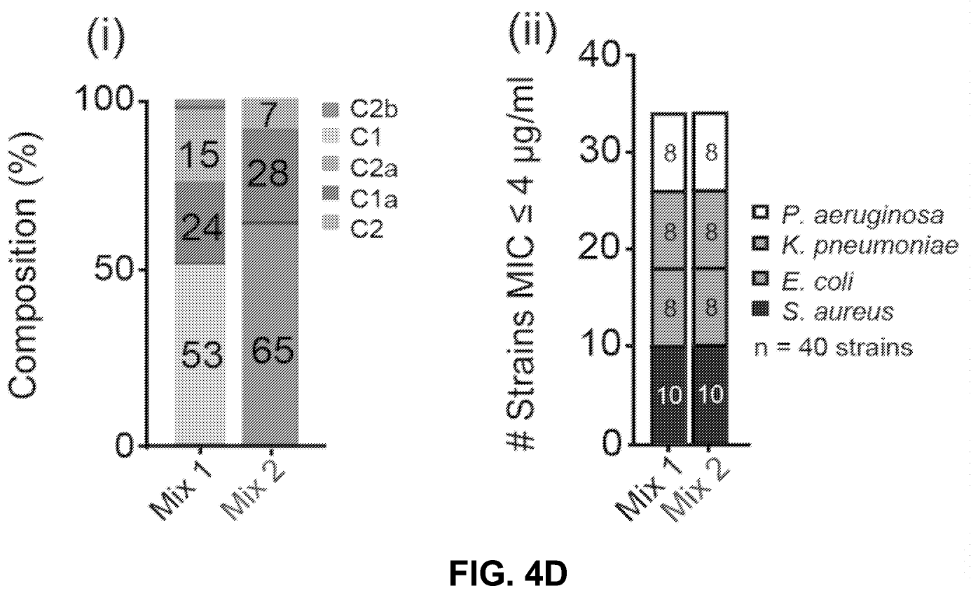

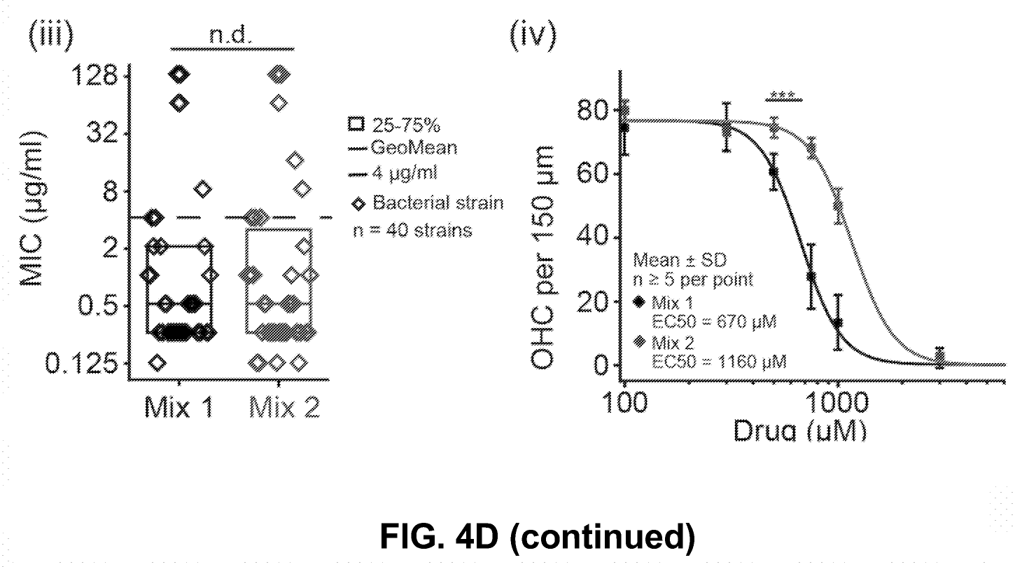

Based on the data presented in FIGS. 2A-D & 3A-E, the inventors conceived the possibility to develop a three-component formulation of gentamicin that is less ototoxic, but has unimpaired antimicrobial activity. First, gentamicin mixture was made without impurities by adding together individual gentamicin C-components (Mix 1) (FIG. 4A). In this example, the percentage composition of this bottle was 53% gentamicin C1, 24% C1a, 15% C2, 6% C2a, and 2% C2b (FIG. 4A), where the mixture totals a 100% of the gentamicin C-components only. This was determined from preliminary analysis of a hospital bottle (data not shown).

Because C2 is more ototoxic than the mixture (P<0.05, F-test, n>5; EC50±SE, Mix 1=670±10 μM, C2=402±38 μM) (FIG. 4B i), the inventors chose to remove C2 from the mixture and kept the ratio of the remaining components the same (FIG. 4B ii). Removal of C2 from the mixture did not affect antimicrobial breadth or potency (P>0.05, Mann Whitney U Test, n=40 strains) (FIG. 4B iii+iv). Next because C1 is reported to be the largest component of hospital bottles, because it shows the lowest antimicrobial potency (FIGS. 2A-D), and because it has the same ototoxicity as the mixture (P>0.05, F-test, n>5; Mix 1=670±10 μM, C1=755±60 μM) (FIG. 4C i), C1 was removed from the mixture. As above, the ratio of the remaining components was kept the same (FIG. 4C ii). Removal of C1 from the mixture did not affect antimicrobial breadth or potency (P>0.05, Mann Whitney U Test, n=40 strains) (FIG. 4C iii+iv). Given that it was thereby demonstrated that C2 and C 1 can both be removed from the mixture without compromising antimicrobial efficacy (FIGS. 4B-C) the inventors next sought to replace the C2 and C1 content with C2b (Mix 2) (FIG. 4D i). Replacement of C2 and C1 with C2b did not impair antimicrobial breadth or potency (P>0.05, Mann Whitney U Test, n=40 strains) (FIG. 4D ii+iii). When examining the ototoxicity of the mixture, it was shown that Mix 2 has reduced ototoxicity (P<0.05, F-test, n>5; EC50±SE, Mix 1=670±10 μM, Mix 2=1148±52 μM (FIG. 4D iv). This data provides evidence that it is possible to reduce ototoxicity without compromising antimicrobial activity by reformulating the ratio of the gentamicin C-components.

Reformulating Hospital Gentamicin to Gentamicin C1a, C2a and C2B

Reformulating hospital gentamicin as a combination of C1a, C2a and C2b makes it less ototoxic. In one embodiment, a formulation of gentamicin for treatment of infections is provided as a mixture of (Table 2):
gentamicin C1a ranging from 10-30%,
gentamicin C2a ranging from 0-30%, and
gentamicin C2b ranging from 40-90%,

whereby the mixture totals a 100% of the gentamicin C-components, defined as C1, C1a, C2, C2a and C2b, only.

In one variation, this formulation is defined such that the combination of the C2a and the C2b is larger than 55%. This reformulation embodiment could be varied as another embodiment by adding to the mixture gentamicin C1 and/or C2 both ranging from 0-10%. In a further variation, this formulation is defined as another embodiment such that the combination of the C2b, C2a and C2 is larger than 55%. In still a further variation, this formulation is defined as another embodiment such that the combination of the C2a and C2 is less than 25%. In still a further variation, this formulation is defined as another embodiment such that the combination of the C2b, C2a and C2 is less than 35%. In yet a further variation of the invention, is to define the formulation as a mixture consisting essentially of C1a, C2a and C2b with the specific ranges outlined herein.

In another embodiment, a formulation of gentamicin for treatment of infections is provided as a mixture of (Table 2):
gentamicin C1a ranging from 10-30%,
gentamicin C2a ranging from 0-20%, and
gentamicin C2b ranging from 50-90%,

whereby the mixture totals a 100% of the gentamicin C-components, defined as C1, C1a, C2, C2a and C2b, only.

In one variation, this formulation is defined such that the combination of the C2a and the C2b is larger than 55%. This reformulation embodiment could be varied as another embodiment by adding to the mixture gentamicin C1 and/or C2 both ranging from 0-10%. In still a further variation, this formulation is defined as another embodiment such that the combination of the C2b, C2a and C2 is larger than 55%. In still a further variation, this formulation is defined as another embodiment such that the combination of the C2a and C2 is less than 25%. In still a further variation, this formulation is defined as another embodiment such that the combination of the C2b, C2a and C2 is less than 35%. In yet a further variation of the invention, is to define the formulation as a mixture consisting essentially of C1a, C2a and C2b with the specific ranges outlined herein.

In yet another embodiment, a formulation of gentamicin for treatment of infections is provided as a mixture of (Table 2):
gentamicin C2b ranging from 90-100% of a total percentage of gentamicin C-components, defined as C1, C1a, C2, C2a and C2b, and impurities ranging from 0-10% in total having the mixture at 100%.

TABLE 2
Reformulation of gentamicin.

| Gentamicin | Embodiment 1 | Embodiment 2 | Embodiment 3 |
|---|---|---|---|
| C1a | 10-30% | 10-30% | |
| C2a | 0-30% | 0-20% | |
| C2b | 40-90% | 50-90% | 90-100% |

These formulations are useful, but not limited to, in pediatric patients for empirical treatment of suspected infection and in the treatment of patients with infections caused by susceptible strains of the following microorganisms: Pseudomonas aeruginosa, Proteus species (indolepositive and indole-negative), Escherichia coli, Klebsiella-Enterobacter-Serratia species, Citrobacter species and Staphylococcus species (coagulasepositive and coagulase-negative).

Method for the Isolation of the Five Major Components of Gentamicin on a Gram Scale: Comparison of the Bactericidal and Ototoxicity of Each Component

Separation of Gentamicin Lot Containing GTC1, GTC2, GTC2a, and GTC1a (Chem Impex Lot#20110415)

a. Salt to free base: 1 g of gentamicin sulfate was dissolved in 20 mL of DI H₂O. Dowex 550A OH anion exchange resin was added to the clear solution until a pH of ˜11 was reached (pH paper). The resin was filtered off and washed with water, and the aqueous filtrate was concentrated to a foamy white solid.

b. Solvent preparation: 500 mL of IPA, 500 mL of 17% NH₄OH, and 1 L of CH₃Cl were sequentially added to a separatory funnel, shaken, and allowed to settle until both layers were clear. Then the bottom layer was collected and used as the eluent. The top aqueous layer was discarded.

c. Chromatographic separation—C1 pure, C2/2a mixture, C1a pure: The free based gentamicin mixture was dry loaded onto silica gel using MeOH followed by evaporation and thorough drying under vacuum before being loaded onto a silica gel chromatography column. The mixture was then eluted with 1:2:1 IPA: CHCl₃:17% NH₄OH, at 40 mL/min over 40 min utilizing a Teledyne ISCO combiflash companion system. In total 96 fractions were collected and visualized by iodine stain on TLC. After visualization it was found that C1 and C2/C2a had eluted. Next the column was flushed with 1:2:1 IPA:CHCl₃:28% NH4OH in order to elute the remaining C1a component of the mixture. The appropriate fractions were collected and reduced under vacuum. Fractions 76-83 contained 107 mg of Gentamicin C1, fractions 86-92 contained 80 mg of C2 and C2a and fractions 6-10 after the solvent swap were collected to give 132 mg of gentamicin C1a. The mixtures of Gentamicin 2 and 2a were separated by preparative HPLC as illustrated in Example 4 below.

d. Preparation of sulfate salts: Free based purified gentamicin component was dissolved in methanol, and 1M H₂SO₄ was added dropwise until the pH of the solution reached 3. The solution was allowed to stand for 10 minutes, and the white precipitate was collected via filtration and washed with methanol. The solid was dried under vacuum.

Separation of Micronomicin Lot Containing GTC2b, and GTC1a (Alfa Aesar, Lot#N17C046)

a. Salt to free base: 5 g of Micronomicin sulfate was dissolved in 100 mL of DI H2O. 63.96 g of Dowex 550A OH anion exchange resin was added to the clear solution until a pH of ˜11 was reached (pH paper). The resin was filtered off and washed with water, and the aqueous filtrate was concentrated to 3.03 g of a foamy white solid.

b. Solvent preparation: 1 L of IPA, 1 L of 17% NH4OH, and 2 L of CH3Cl were sequentially added to a separatory funnel, shaken, and allowed to settle until both layers were clear. Then the bottom layer was collected and used eluent. The top aqueous layer was discarded. In the case of the 1:2:1 IPA:CHCl3:28% NH4OH eluent, the same procedure as above was followed, but with 28% NH4OH in place of the 17% NH4OH.

c. Chromatographic separation—C2b and C1a pure: The free based micronomicin mixture was dry loaded onto silica gel using MeOH. The mixture was then eluted with 1:2:1 IPA:CHCl3:17% NH4OH, at 40 mL/min utilizing a Teledyne ISCO combiflash companion system until it was found that C2b had completely eluted. ˜90 20 mL fractions were collected and visualized by iodine stain on TLC. Next, the column was flushed with 1:2:1 IPA:CHCl3:28% NH4OH in order to elute the remaining C1a component of the mixture. ˜120 20 mL fractions were collected. The appropriate fractions were collected and reduced under vacuum to give 1.98 g of C2b and 0.68 g of C1a as white foams.

Large Scale Separation of Micronomicin—GTC2b and GTC1a

a. Salt to Free Base: 25.22 g of Micronomicin sulfate was dissolved in 500 mL of water and Dowex 550A resin was added and stirred overnight until a pH of ˜11 was reached. The resin was filtered and the volatiles removed to give 17.81 g of a white foam.

b. Chromatographic separation C2b and C1a pure This mixture was dry packed on to silica gel and a similar procedure as for the small scale was followed using a 330 g column and a flow rate of 100 mL/min. 10.6 g of the Gentamicin C2b free base and 2.5 g of the Gentamicin C1a free base were recovered.

c. Example Procedure for the preparation of the sulfate salt: The pure free base gentamicin component was dissolved in methanol, and 1M H₂SO₄ was added dropwise until the pH of the solution reached 3. The solution was allowed to stand for 10 min, and the white precipitate was collected via filtration and washed with methanol. The solid was thoroughly dried under vacuum. Thus 1.98 g of gentamicin C2b free base afforded 2.10 g of C2b sulfate. Gentamicin C1a (0.68 g) provided 1.04 g of C1a sulfate as a white powder.

Isolation of Gentamicin C2 and C2a from the Mixture by Preparative HPLC

The mixture of gentamicin C2 and C2a obtained in Example 1 above was separated by multiple injections of 200 mg each (45 injections, 9 grams of mixture in total). The conditions used were as follows:

Column: X-Bridge Prep C18, 10 μm OBD 50×250 mm

Buffer A and B: 97% dH₂O+3% MeOH+0.2% TFA

Gradient: 50% B Isocratic, 60 min, 60 mL/minute flow rate

Loading: 200 mg dissolved in 3.0 mL dH₂O

Using this method, gentamicin C2 (RT=11.42 min) and C2a (RT=14.83 min) were isolated in pure form and converted to the sulfate salt as described in previous examples. The salts were thoroughly dried under vacuum.

What is claimed is:

1. A formulation of gentamicin for treatment of infections, comprising a mixture of:
gentamicin C1a ranging from 10-30%, gentamicin C2a ranging from 0-30%, and gentamicin C2b ranging from 40-90%, wherein the mixture totals a 100% of the gentamicin C-components, defined as C1, C1a, C2, C2a and C2b only.

2. The formulation as set forth in claim 1, wherein the combination of the C2a and the C2b is larger than 55%.

3. The formulation as set forth in claim 1, wherein the mixture further comprises C1 ranging from 0-10%.

4. The formulation as set forth in claim 1, wherein the mixture further comprises C2 ranging from 0-10%.

5. The formulation as set forth in claim 4, wherein the combination of the C2 and the C2a is less than 25%.

6. A formulation of gentamicin for treatment of infections, comprising a mixture of:
gentamicin C2b ranging from 90-100% of a total percentage of gentamicin C-components, defined as C1, C1a, C2, C2a and C2b, and impurities ranging from 0-10% in total having the mixture at 100%.
